(19) United States
(12) Patent Application Publication
(10) Pub. No.: US 2024/0225770 A1
(43) Pub. Date: Jul. 11, 2024

(54) ORTHOPEDIC DEVICE CADDIES AND KITS

(71) Applicant: INTERNATIONAL LIFE SCIENCES, LLC d/b/a ARTELON, Marietta, GA (US)

(72) Inventors: Justin C. SLUDER, Dallas, GA (US); Lauren Chase THORNBURG, Cumming, GA (US); David Brett CAIN, Marietta, GA (US)

(73) Assignee: INTERNATIONAL LIFE SCIENCES, LLC d/b/a ARTELON, Marietta, GA (US)

(21) Appl. No.: 18/486,749

(22) Filed: Oct. 13, 2023

Related U.S. Application Data
(60) Provisional application No. 63/479,521, filed on Jan. 11, 2023.

(51) Int. Cl. A61B 50/31; A61B 17/86

(52) U.S. Cl. CPC A61B 50/31; A61B 17/865; A61B 2050/3008

(57) ABSTRACT

Orthopedic device caddies and kits including orthopedic device caddies. In one example, the orthopedic device caddy includes a caddy body, an elongated opening extending into the caddy body including an alignment portion and an orthopedic device retention portion. The orthopedic device retention portion of the elongated opening holds an orthopedic device having an orthopedic device engagement feature. The alignment portion of the elongated opening aligns an instrument engagement feature of an orthopedic instrument relative to the orthopedic device engagement feature.

### CROSS-REFERENCE TO RELATED FILINGS

This patent application claims priority to and the benefit of the filing date of U.S. provisional patent application Ser. No. 63/479,521 filed Jan. 11, 2023 for “Orthopedic Device Caddies and Kits,” the entire contents of which are hereby incorporated by this reference.

### RELATED FIELDS

Orthopedic device caddies and kits including orthopedic device caddies.

### BACKGROUND

Orthopedic procedures often require the presence of numerous orthopedic instruments, implants, and other orthopedic devices in the sterile surgical field. Some of the implants and other devices required for a particular orthopedic procedure can be quite small, such as small screws, eyelets, and other orthopedic devices. In some instances, caddies are used to hold these devices so that they can be easily accessed during the orthopedic procedure. There are drawbacks to existing caddies; however, and there remains room for improvement.

### SUMMARY

This patent describes improved orthopedic device caddies and kits for the same.

### First Example

In one example, an orthopedic kit includes an orthopedic device having an orthopedic device engagement feature, an orthopedic instrument having an elongated instrument shaft and an instrument engagement feature, and an orthopedic device caddy. In this example, the orthopedic device caddy has a caddy body and an elongated opening extending into the caddy body. The elongated opening has an alignment portion extending along a proximal segment of the elongated opening, and an orthopedic device retention portion extending along a segment of the elongated opening distal to the proximal segment. The orthopedic device retention portion is configured to hold the orthopedic device, and the alignment portion is configured to receive a portion of the instrument shaft and align the instrument engagement feature relative to the orthopedic device engagement feature.

In this example, the kit may also include a sterile package containing the orthopedic device, the orthopedic instrument, and the orthopedic device caddy.

In this example, the alignment portion may be at least three times a length of the orthopedic device, or may be at least twice a length of the orthopedic device.

In this example, an outer diameter of the portion of the instrument shaft may be less than 1 mm smaller than an inner diameter of the alignment portion of the elongated opening, or may be less than 0.5 mm smaller than an inner diameter of the alignment portion of the elongated opening.

In this example, the elongated opening may extend into the caddy body at a non-perpendicular and non-parallel angle relative to a planar face of the caddy body.

In this example, the caddy body may be configured to allow visualization of the orthopedic device in the orthopedic device retention portion of the elongated opening.

In this example, the caddy body may be at least partially translucent.

In this example, the caddy body may include a visualization opening extending into the caddy body proximate the orthopedic device retention portion of the elongated opening.

In this example, the caddy body may include a vent opening extending into the caddy body to the orthopedic device retention portion.

In this example, the caddy body may include at least one additional opening extending into the caddy body configured to retain an additional orthopedic device.

In this example, the additional orthopedic device may be an orthopedic screw.

In this example, the orthopedic device may include a first orthopedic device and a second orthopedic device, and the orthopedic device retention portion may include a first orthopedic device retention portion and a second orthopedic device retention portion spaced from the first orthopedic device retention portion along the elongated opening, with the first orthopedic device retention portion configured to hold the first orthopedic device and the second orthopedic device retention portion configured to hold the second orthopedic device.

In this example, the first orthopedic device retention portion may distal to the alignment portion and the second orthopedic device retention portion may be either proximal to or in the alignment portion.

In this example, the second orthopedic device may be cannulated and configured to receive the elongated instrument shaft of the orthopedic instrument such that the elongated instrument shaft extends through the cannulation of the second orthopedic device.

In this example, the caddy body may further include a port extending into the caddy body proximate the second orthopedic device retention portion.

In this example, the second orthopedic device may be physically manipulable through the port when the second orthopedic device is located in the second orthopedic device retention portion.

### Second Example

In another example, an orthopedic device caddy includes a caddy body and an elongated opening extending into the caddy body, the elongated opening including an alignment portion extending along a proximal segment of the elongated opening, and an orthopedic device retention portion extending along a segment of the elongated opening distal to the proximal segment. The orthopedic device retention portion is configured to hold an orthopedic device having an orthopedic device engagement feature. The alignment portion is configured to align an instrument engagement feature of an orthopedic instrument relative to the orthopedic device engagement feature.

In this example, the alignment portion may be at least three times the length of the orthopedic device retention portion, or may be at least twice the length of the orthopedic device retention portion.

In this example, the elongated opening may extend into the caddy body at a non-perpendicular and non-parallel angle relative to a planar face of the caddy body.

In this example, the caddy body may be configured to allow visualization of the orthopedic device in the orthopedic device retention portion of the elongated opening.

In this example, the caddy body may include at least one additional opening extending into the caddy body configured to retain an additional orthopedic device.

### Third Example

In another example, an orthopedic kit includes an orthopedic device including an orthopedic device engagement feature, an orthopedic screw, an orthopedic instrument including an elongated instrument shaft and an instrument engagement feature, and an orthopedic device caddy. The orthopedic device caddy includes a caddy body and an elongated opening extending into the caddy body, and at least one additional opening extending into the caddy body configured to retain the orthopedic screw. The elongated opening includes an alignment portion extending along a proximal segment of the elongated opening and an orthopedic device retention portion extending along a segment of the elongated opening distal to the proximal segment. The orthopedic device retention portion is configured to hold the orthopedic device. The alignment portion is configured to receive a portion of the instrument shaft and align the instrument engagement feature relative to the orthopedic device engagement feature, the alignment portion being at least twice a length of the orthopedic device.

### Fourth Example

In another example an orthopedic system includes an orthopedic device caddy. The orthopedic device caddy has a caddy body with an elongated opening extending into the caddy body from a proximal end to a distal end. The elongated opening has a first portion and a second portion that is wider than the first portion. A first orthopedic device is removably positioned in the first portion of the elongated opening, and a second orthopedic device is removably positioned in the second portion of the elongated opening.

In this example the second orthopedic device may be cannulated, with the cannulation extending from a proximal end of the second orthopedic device to a distal end of the second orthopedic device.

In this example the first and second orthopedic devices may be removably positioned in the first and second portions of the elongated opening. The distal end of the second orthopedic device may be spaced apart from a proximal end of the first orthopedic device.

In this example the orthopedic device caddy may also include a movable stop movable between a blocking position and a non-blocking position. When the movable stop is in the blocking position, the movable stop may be positioned to obstruct at least partially the elongated opening such that the second orthopedic device cannot move fully out of the second portion of the elongated opening. When the movable stop is in the non-blocking position, the movable stop may be positioned such that it does not prevent the second orthopedic device from moving fully out of the second portion of the elongated opening.

In this example when the movable stop is in the blocking position it may extend further into the elongated opening than in the non-blocking position.

In this example the elongated opening may include a third portion proximal to the second portion and wider than the second portion. The movable stop may be located at the third portion of the elongated opening.

In this example the movable stop may be biased to the blocking position by a resilient member.

In this example the movable stop may be a movable ball.

In this example the system may further include an orthopedic instrument. The instrument may include an elongated instrument shaft. The instrument may be sized and shaped such that insertion of the orthopedic instrument into the elongated opening of the caddy body engages the first and second orthopedic devices to the elongated instrument shaft and moves the movable stop to the non-blocking position.

### BRIEF DESCRIPTION OF FIGURES

FIG. 1 is an example of an orthopedic device caddy.

FIGS. 2-3 are photographs of the orthopedic device caddy of FIG. 1, shown loaded with orthopedic screws and eyelets.

FIGS. 4-6 are photographs showing use of the orthopedic device caddy of FIG. 1 to load an eyelet onto an instrument.

FIGS. 7-8 are photographs showing use of the orthopedic device caddy of FIG. 1 to load a screw onto an instrument.

FIGS. 9-16 show another example of an orthopedic device caddy.

FIGS. 17-28 show another example of an orthopedic device caddy.

### DETAILED DESCRIPTION OF FIGURES

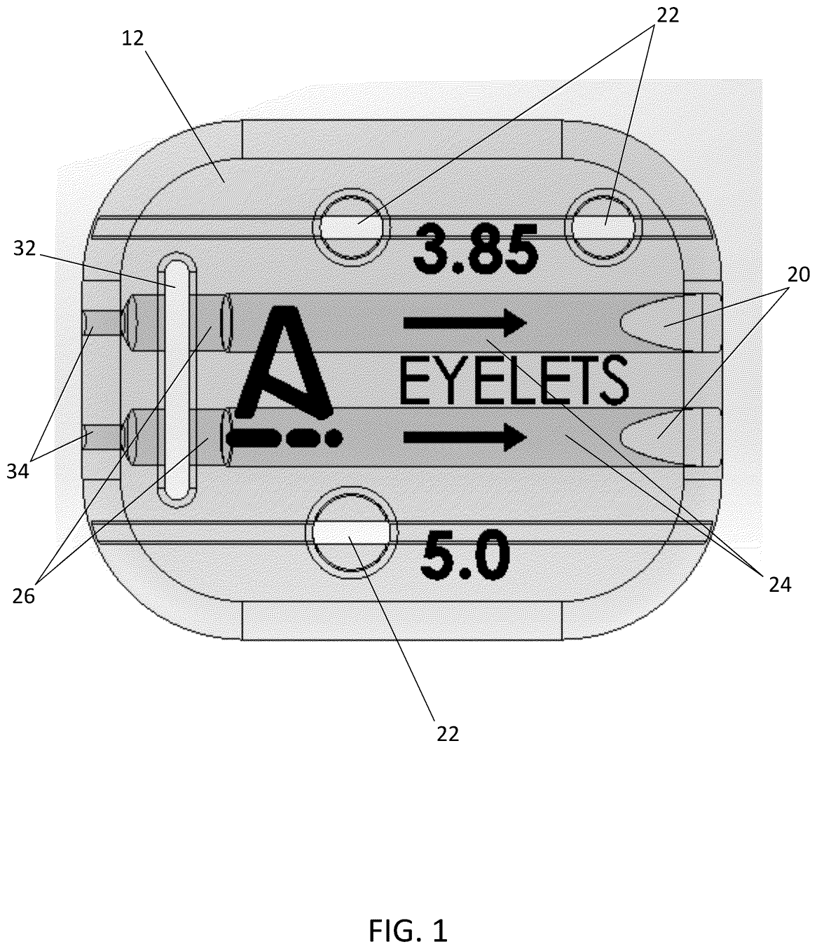

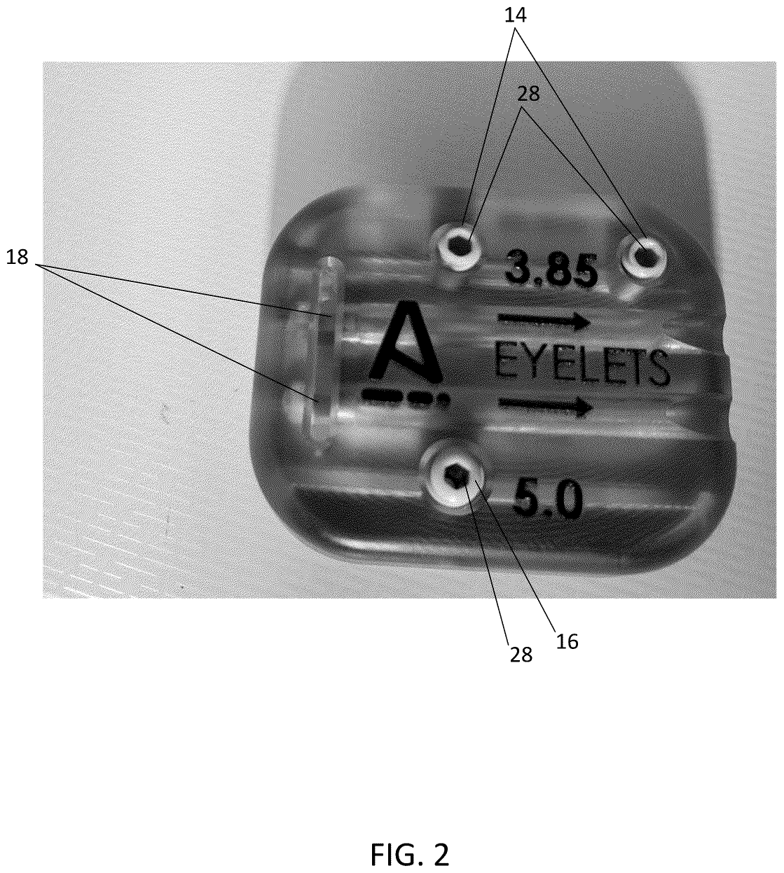

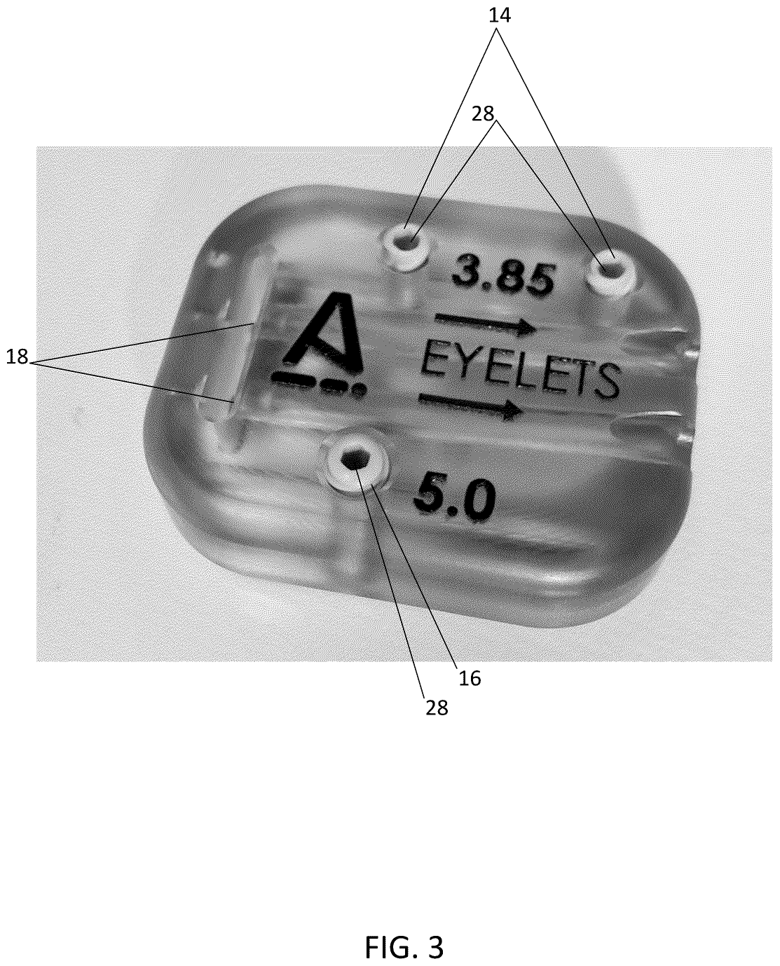

### FIGS. 1-8

FIGS. 1-3 show an example of an orthopedic device caddy. The caddy includes a caddy body 12 and several openings extending into the caddy body for holding various orthopedic devices.

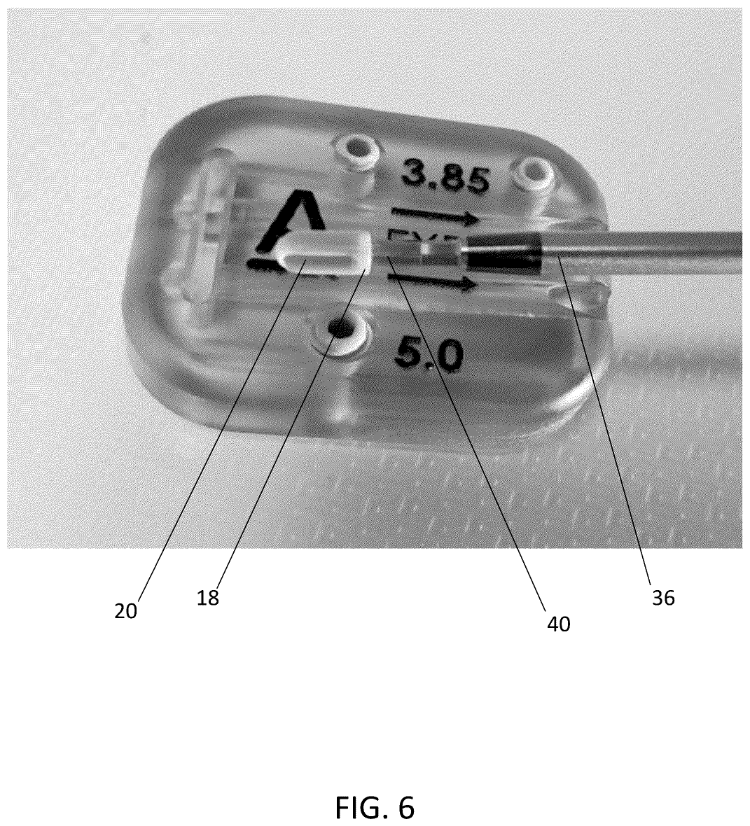

In this example, the orthopedic devices are two sizes of orthopedic screws 14, 16 and a pair of orthopedic eyelets 18. As shown in FIG. 6, the eyelets 18 include an opening 30 extending through it, which allows the eyelet to receive one or more suture strands, suture tapes, or surgical meshes (such as a Flexband® biomaterial available from Artelon) used in certain orthopedic procedures. In other examples, the orthopedic device caddy may be configured to hold other types and numbers of orthopedic devices.

The screws 14, 16 and eyelets 18 may each include an engagement feature (e.g. the hexagonal openings 28 in the heads of the screws 14, 16) that facilitate engaging the orthopedic device to a corresponding engagement feature of an orthopedic instrument. The screws 14, 16, and eyelets 18 may all have the same engagement feature so that a single instrument may engage all of them or may have different engagement features for engagement with different instruments.

In the example of FIGS. 1-3, the openings 20 are configured to hold orthopedic eyelets 18 and the openings 22 are configured to hold the orthopedic screws 14, 16.

The openings 20 configured to hold orthopedic eyelets 18 are elongated openings. Each opening 20 includes an alignment portion 24 extending along a proximal segment of the opening 20 and an orthopedic device retention portion 26 extending along a segment of the opening 20 that is distal to the proximal segment. The orthopedic device retention portion 26 is configured to hold the orthopedic device (such as the eyelet 18 or other relatively small orthopedic device component). In the particular example shown, the eyelet 18 freely rests at the bottom of the opening 20. In other implementations, the size, shape, and configuration of the eyelet 18 and opening 20 may be adjusted to provide a light press fit between the two.

The alignment portion 24 is configured to align an instrument engagement feature of an orthopedic instrument relative to the orthopedic device engagement feature of the orthopedic device located in the retention portion 26. This allows relatively easy loading of a small orthopedic device onto an instrument without risk of droppage or other mishandling of the small orthopedic device in the sterile surgical field.

In the example of FIGS. 1-3, the alignment portion 24 is relatively long and has an inner diameter closely matched to the outer diameter of the portion of the instrument shaft that is inserted into the opening 20, thereby facilitating bringing an engagement feature of the instrument into alignment with the corresponding engagement feature of the orthopedic device in the opening when the instrument is inserted into the opening 20. In some implementations, the alignment portion 24 is at least two times the length of the orthopedic device retaining portion 26 and the orthopedic device 18 therein. In some implementations, the alignment portion 24 is at least three times the length of the orthopedic device retaining portion 26 and the orthopedic device 18 therein. In some implementations, the outer diameter of the portion of the instrument shaft is less than 1 mm smaller than an inner diameter of the alignment portion of the elongated opening. In some implementations, the outer diameter of the portion of the instrument shaft is less than 0.5 mm smaller than an inner diameter of the alignment portion of the elongated opening.

In the example of FIGS. 1-3, the elongated openings 20 extend into the caddy body 12 at a non-perpendicular and non-parallel angle relative to a planar face of the caddy body 12. The angle of the elongated openings 20 allows for maximizing their length relative to the caddy body 12. The angle of the elongated openings 20 also facilitates retaining the orthopedic devices 18 in the bottom of the openings 20 so that they will not accidentally fall out of the openings 20 as the caddy is handled.

In the example of FIGS. 1-3, the caddy body 12 is configured to allow visualization of the orthopedic devices 18 retained at the bottom of openings 20. In this example, the caddy body 12 is partially translucent (e.g. formed of a partially translucent plastic material), thereby allowing visualization. Additionally, in this example, the caddy body 12 includes a visualization opening 32 extending into the body proximate the retention portion 26 of the openings 20, thereby further allowing visualization of the orthopedic devices 18 in the openings 20.

In the example of FIGS. 1-3, the caddy body 12 also includes vent openings 34 that extend to the openings 20, 22, thereby facilitating the penetration of and venting of a sterilization gas into the openings 20, 22 and around the orthopedic devices 14, 16, 18 held therein.

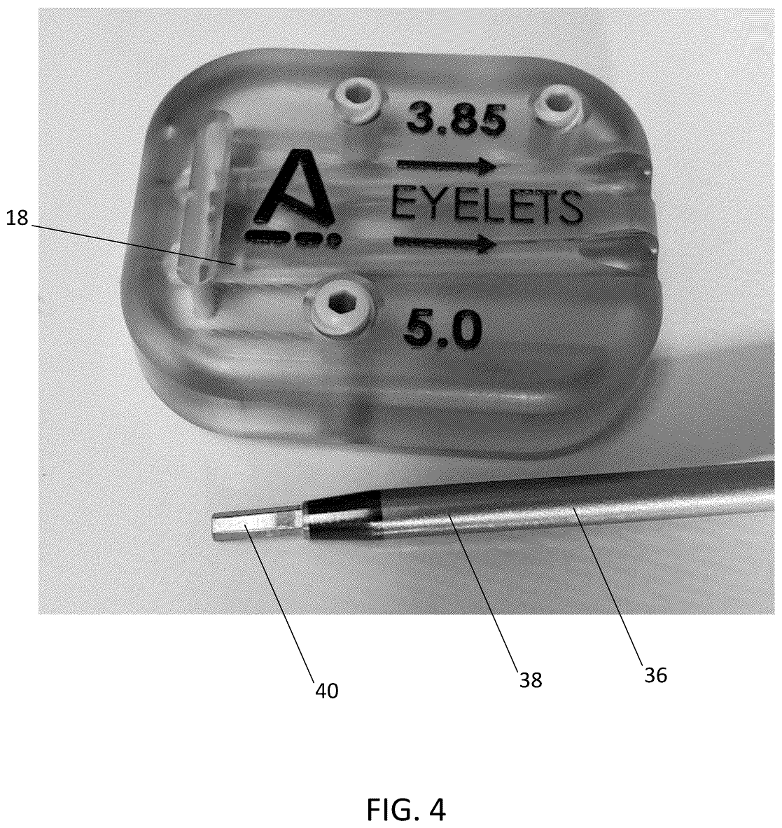

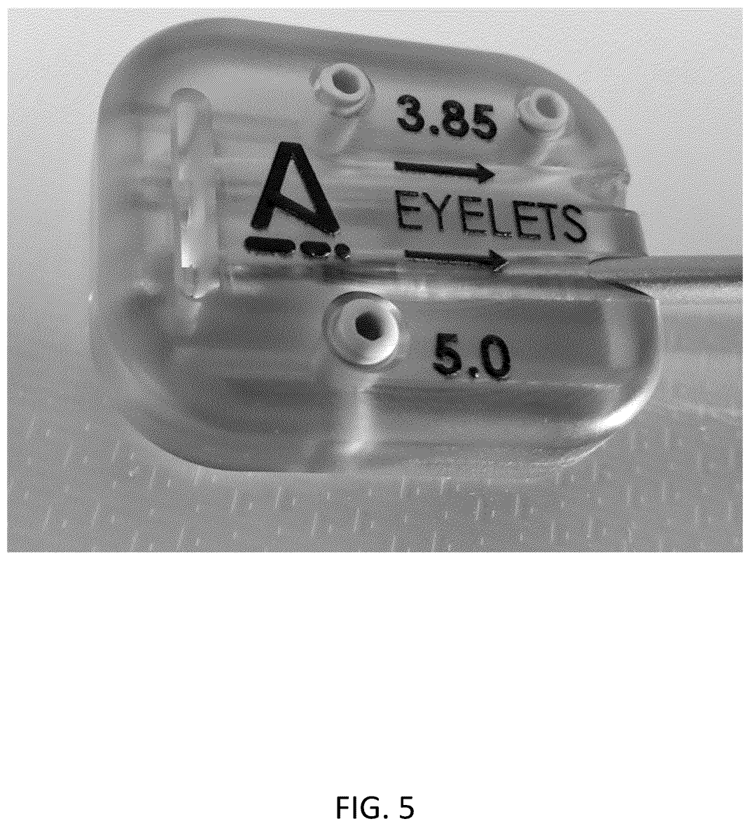

FIGS. 4-6 show one example of loading an orthopedic device 18 from an opening 20 in the screw caddy onto an instrument 36. FIG. 4 shows the instrument 36 prior to insertion into an opening 20 in the screw caddy. The instrument 36 includes an elongated instrument shaft 38 and an engagement feature 40 at a distal end of the instrument shaft 38. FIG. 5 shows a portion of the instrument shaft 38 inserted into the opening 20 in the screw caddy such that the engagement feature 40 on the instrument shaft 38 is brought into alignment with and engagement with an engagement feature (not shown) on the orthopedic device 18 held by the screw caddy in the retention portion 26 of opening 20. As discussed above, the length and diameter of the alignment portion 24 of the opening 20 may be configured to help easily guide the instrument 36 into engagement with the orthopedic device 18. FIG. 6 shows the orthopedic device 18 removed from the opening 20 by the instrument 36 and ready for use in the surgical procedure.

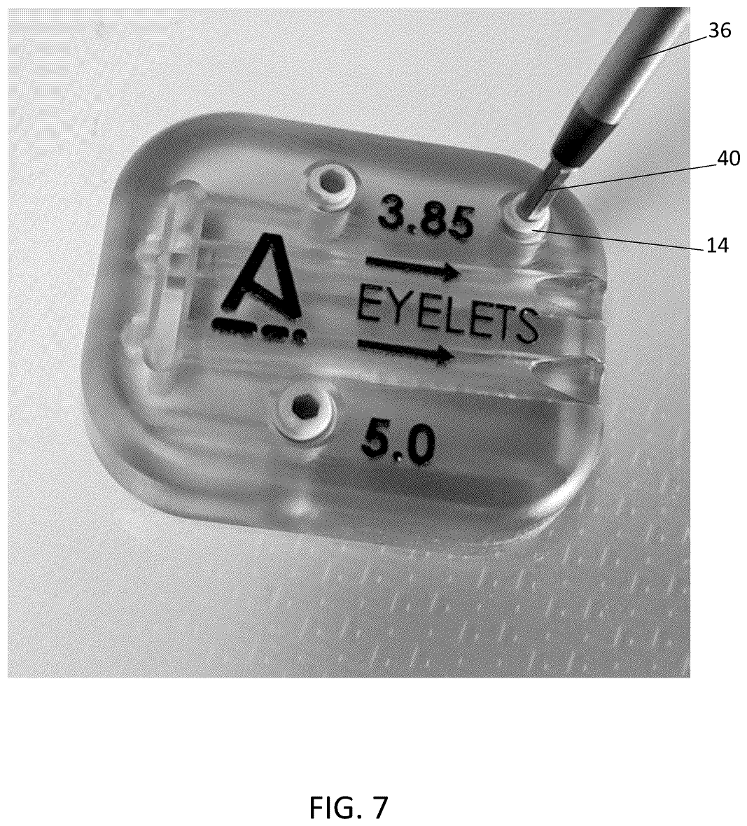

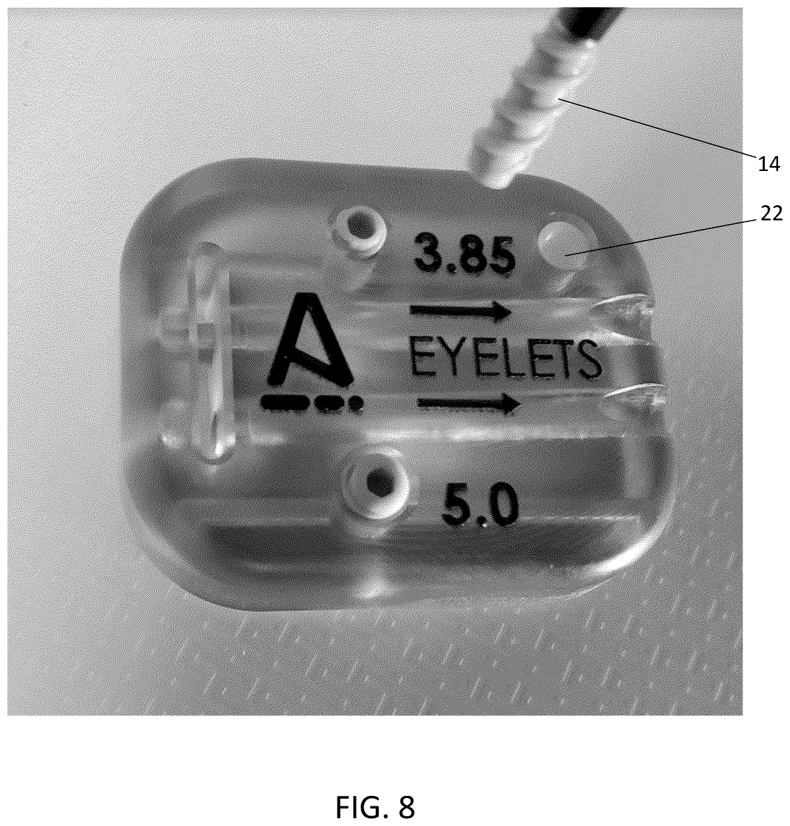

FIGS. 7-8 show one example of loading an orthopedic device 14 from an opening 22 in screw caddy onto the instrument 36. FIG. 7 shows the engagement feature 40 of instrument 36 brought into engagement with an engagement feature on the orthopedic device 14, and FIG. 8 shows the orthopedic device 14 removed from the opening 22 by the instrument 36.

The orthopedic device caddy, orthopedic devices, and instrument(s) may all be provided as part of an orthopedic kit. In some implementations, the orthopedic device caddy, orthopedic devices, and instrument(s) may be packaged together in a sterile package. In some implementations, at least some of the kit components may be packaged in separate sterile packages.

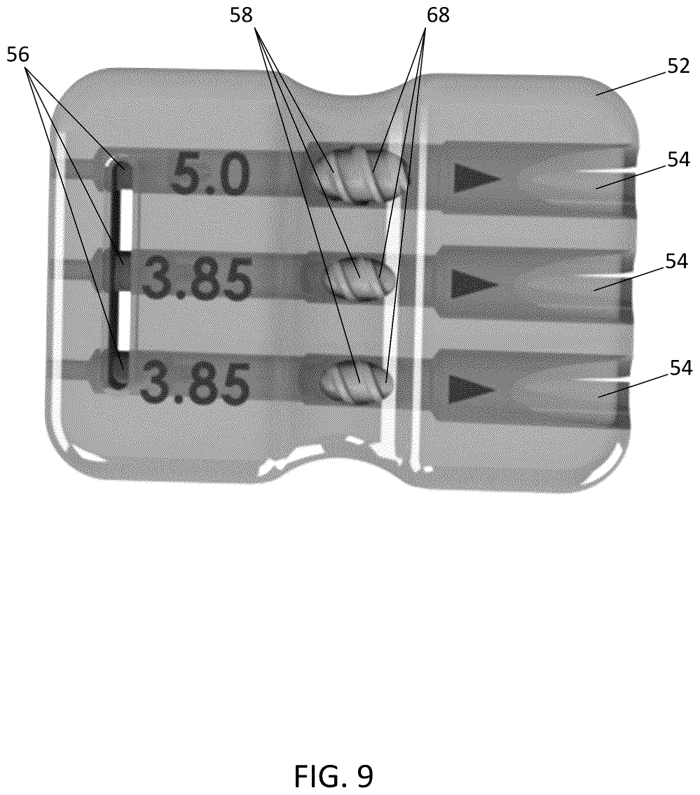

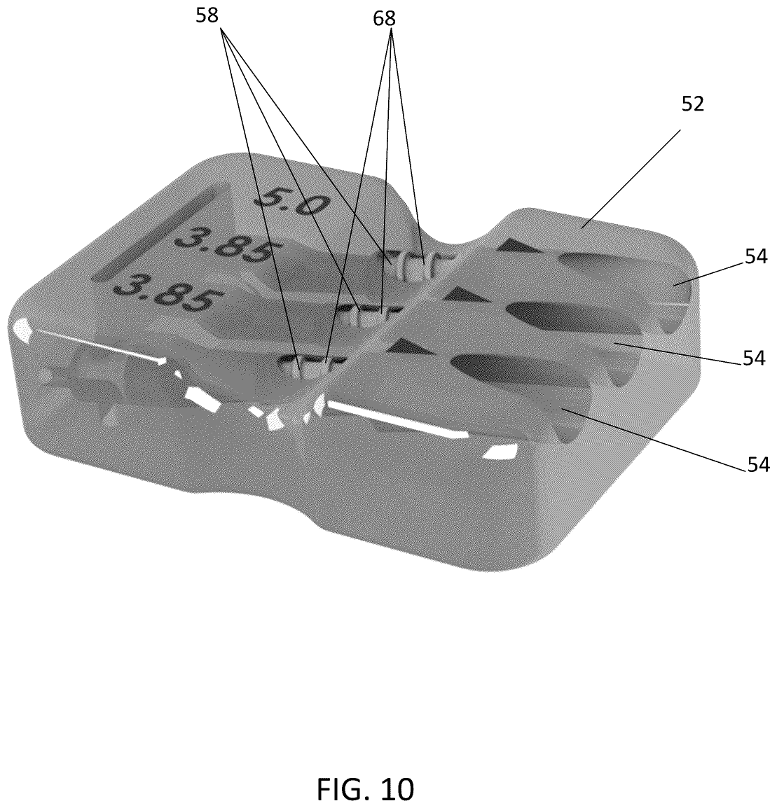

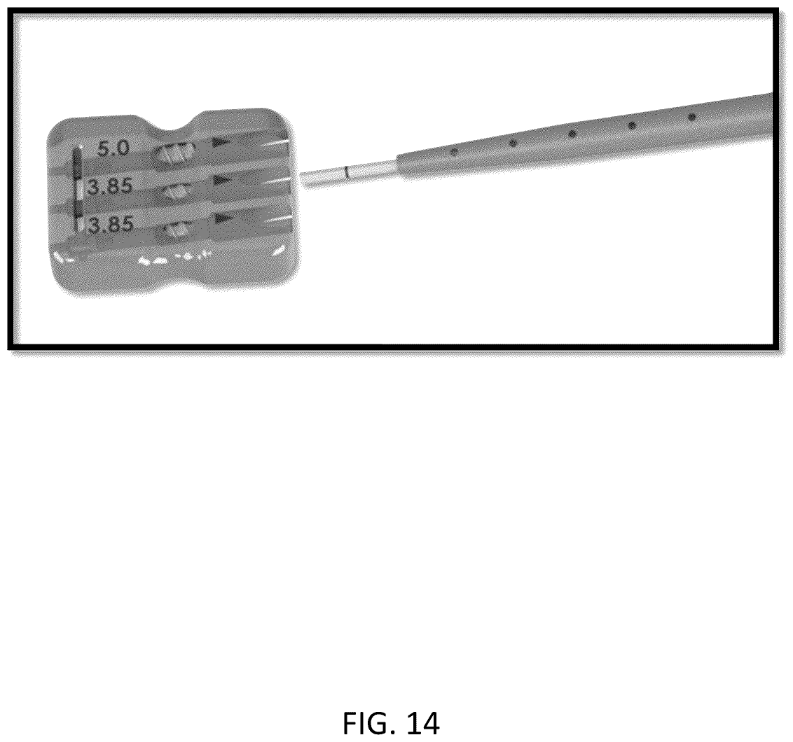

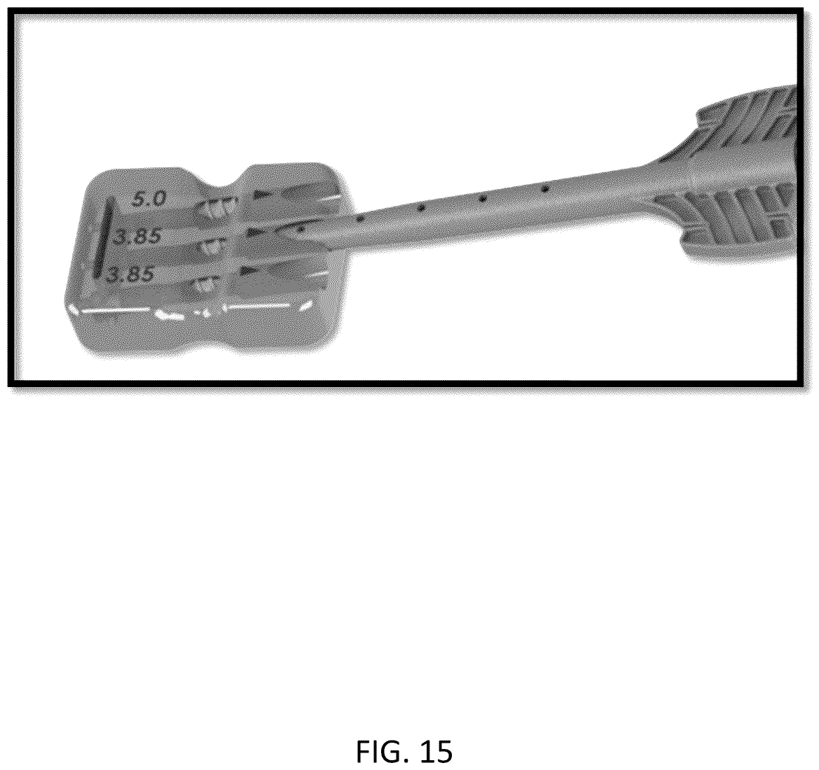

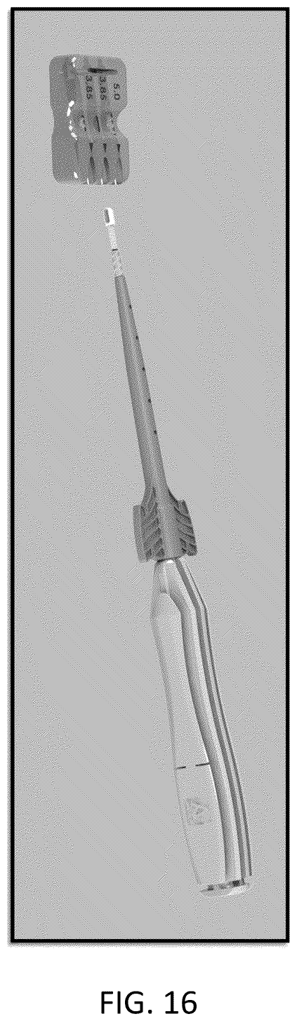

### FIGS. 9-16

FIGS. 9-16 show another example of an orthopedic device caddy. In this example, the openings extending into the caddy body are each configured to hold multiple orthopedic devices, and multiple orthopedic devices can be simultaneously loaded onto an instrument by inserting the instrument's shaft into the opening.

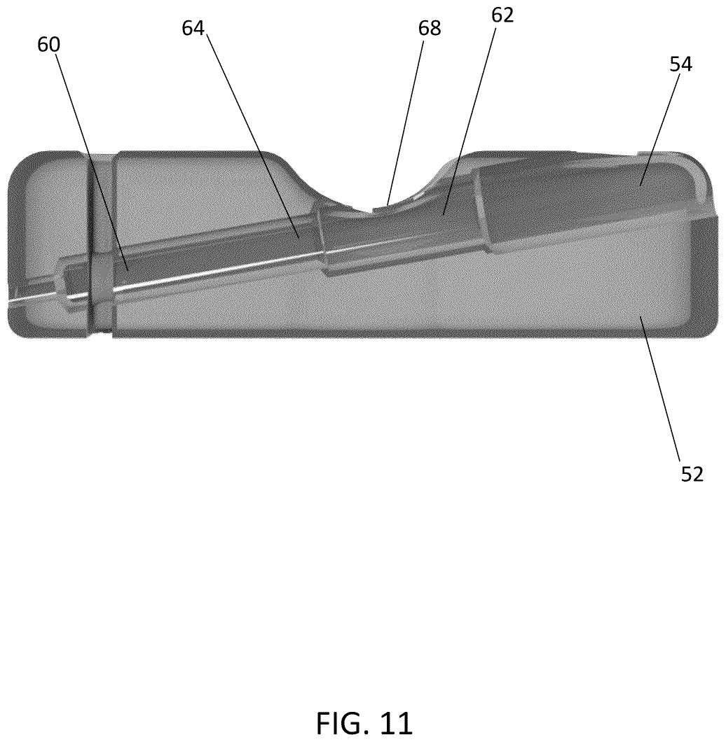

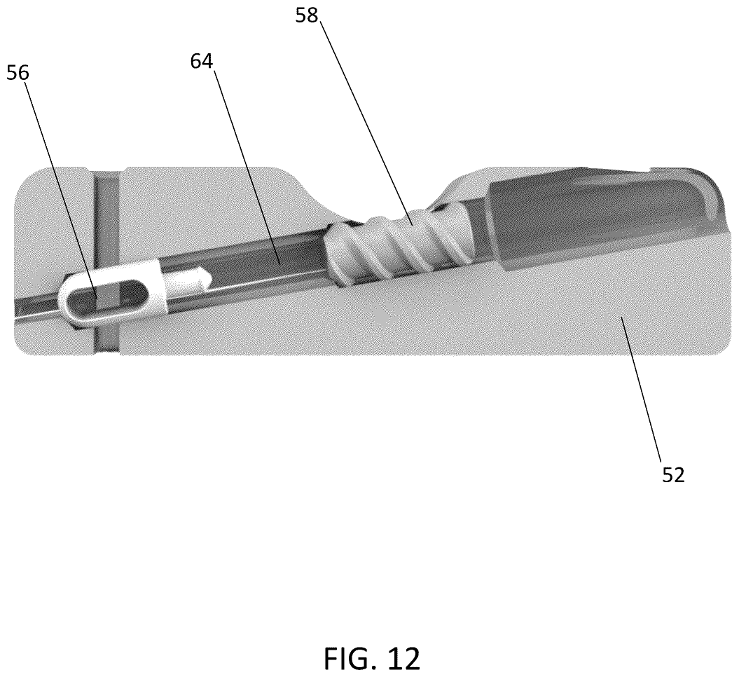

In the example of FIGS. 9-16, the caddy body 52 has three elongated openings 54 extending into the caddy body 52, each opening 54 configured to receive both a first orthopedic device 56 and a second orthopedic device 58. As shown in, for instance, FIGS. 11 and 12, each opening 54 has a first orthopedic device retention portion 60 configured to receive the first orthopedic device 56 and a second orthopedic device retention portion 62 configured to receive the second orthopedic device 58. The first and second orthopedic device retention portions 60, 62 are spaced apart from one another along the elongated opening 54, with part of an alignment portion 64 of the opening 54 separating the two. As shown in this example, the first orthopedic device retention portion 60 is distal to the alignment portion 64, and the second orthopedic device retention portion 62 is in the alignment portion 64. In other examples, the second orthopedic device retention portion may be located at the proximal end of the opening such that it is also proximal to the entire alignment portion of the opening.

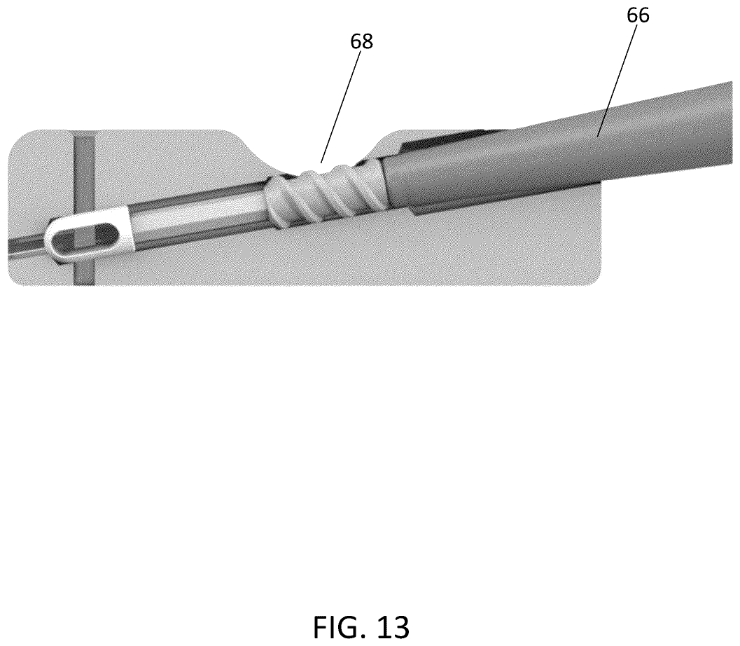

In the example of FIGS. 9-16, the first orthopedic device 56 is the same as or similar to the eyelet 18 discussed above in the context of FIGS. 1-8, and includes an engagement feature for engaging a corresponding feature on a distal end of an orthopedic instrument 66 as shown in for instance FIGS. 12 and 13. In the example of FIGS. 9-16, the second orthopedic device 58 is a cannulated device (e.g. a cannulated screw) that is configured to receive the elongated shaft of the orthopedic instrument 66 such that the elongated instrument shaft extends through the cannulation of the second orthopedic device 58 when the instrument 66 is inserted into the opening 54. As can be seen in FIGS. 12 and 13, when the instrument 66 is inserted into the opening 54, the elongated shaft of the instrument 66 will extend through the cannulation of the second orthopedic device 58 and engage the engagement feature on the first orthopedic device 56, thereby mounting both of the orthopedic devices 56, 58 onto the instrument 66 simultaneously.

As shown in FIG. 12, the caddy body 52 includes a port 68 extending into the caddy body 52 proximate the second orthopedic device retention portion 62. The port 68 allows for physical manipulation of the second orthopedic device 58 while it is located in the second orthopedic device retention portion 62. For example, although not shown in the figures, the second orthopedic device may have a cannulation that is hexagonal in cross section to match a hexagonal cross section of the instrument 66 shaft, and it may be desirable to manipulate the second orthopedic device 58 (e.g. use a finger to hold it still) while the instrument 66 is rotated in the opening 54 to line up the hexagonal cross sections of the instrument and device. The port 68 may be shaped, sized, and otherwise configured for a gloved finger to physically contact and hold the device still in the device retention portion.

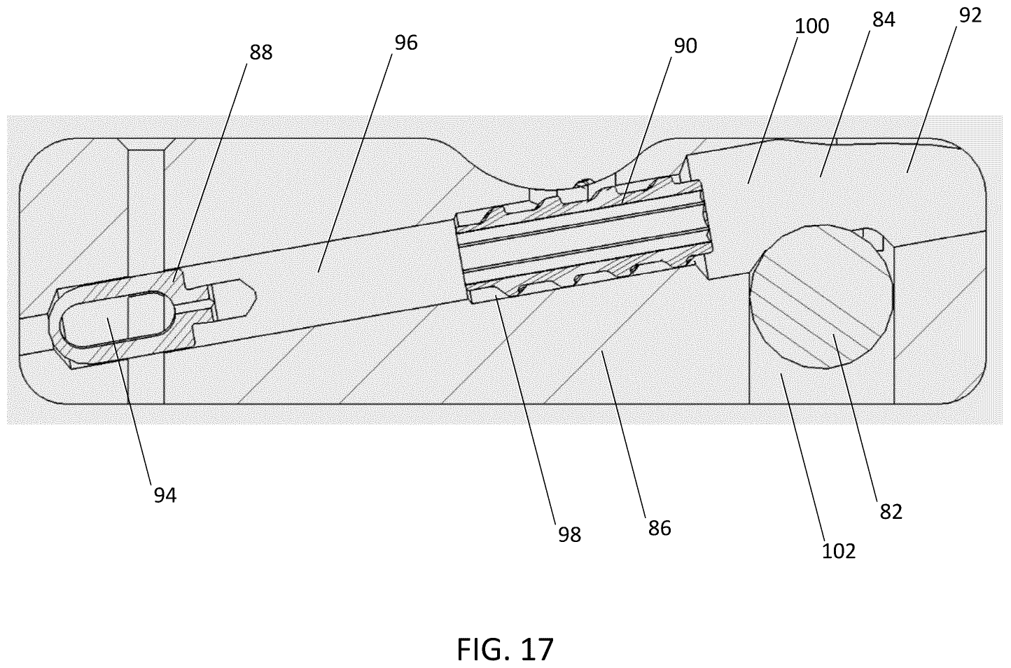

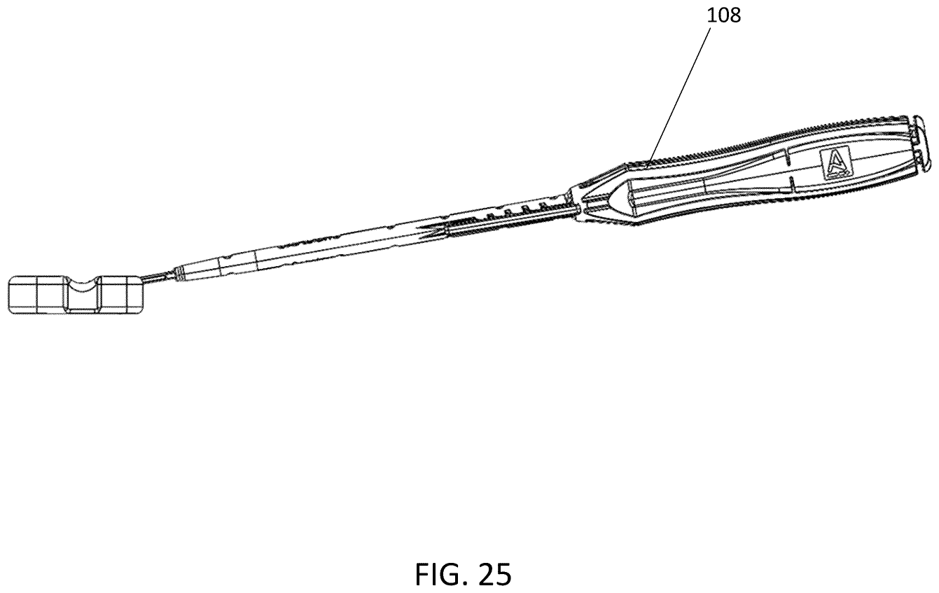

### FIGS. 17-28

FIGS. 17-28 show another example of an orthopedic device caddy. In this example the caddy includes movable stops 82 that partially obstruct the openings 84 in the caddy body 86 when the movable stops 82 are in blocking positions. The movable stops 82 prevent unintentional dislodgement and ejection of the orthopedic devices 88, 90 held in the caddy.

FIG. 17 shows the orthopedic device caddy of this example in cross section. As shown in FIG. 17 the elongated opening 84 extends into the caddy body 86 from a proximal end 92 of the elongated opening 84 to a distal end 94 of the elongated opening 84. The elongated opening 84 includes a first portion 96, second portion 98, and third portion 100. The third portion 100 is wider than the second portion 98, which is wider than the first portion 96, with the widths stepping down at shoulders between each of the portions.

The elongated opening 84 and how its width changes in this example is similar to the elongated opening 54 in the example of FIGS. 9-16. The first portion 96 of the elongated opening 84 includes a retention portion in which the first orthopedic device 88 is removably positioned (similar to retention portions 26 and 60 in earlier examples), and also includes an alignment portion (similar to alignment portions 24 and 64 shown in earlier examples). The second portion 98 of elongated opening 84 constitutes a retention portion in which the second orthopedic device 90 is removably positioned (similar to retention portion 62 in an earlier example).

As in the earlier example of FIGS. 9-16, the first orthopedic device 88 may be an eyelet or another type of device, and the second orthopedic device 90 may be a cannulated screw or another type of device, with the distal end of the second orthopedic device 90 spaced apart from the proximal end of the first orthopedic device 88 when the devices 88, 90 are removably positioned in the first and second portions 96, 98 of the elongated opening 84.

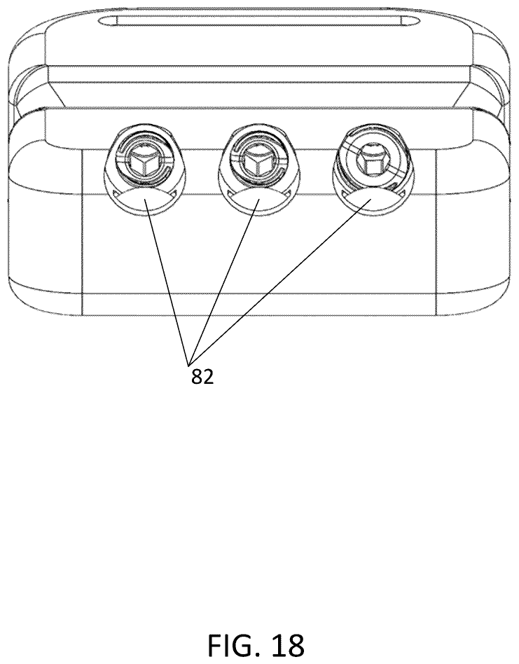

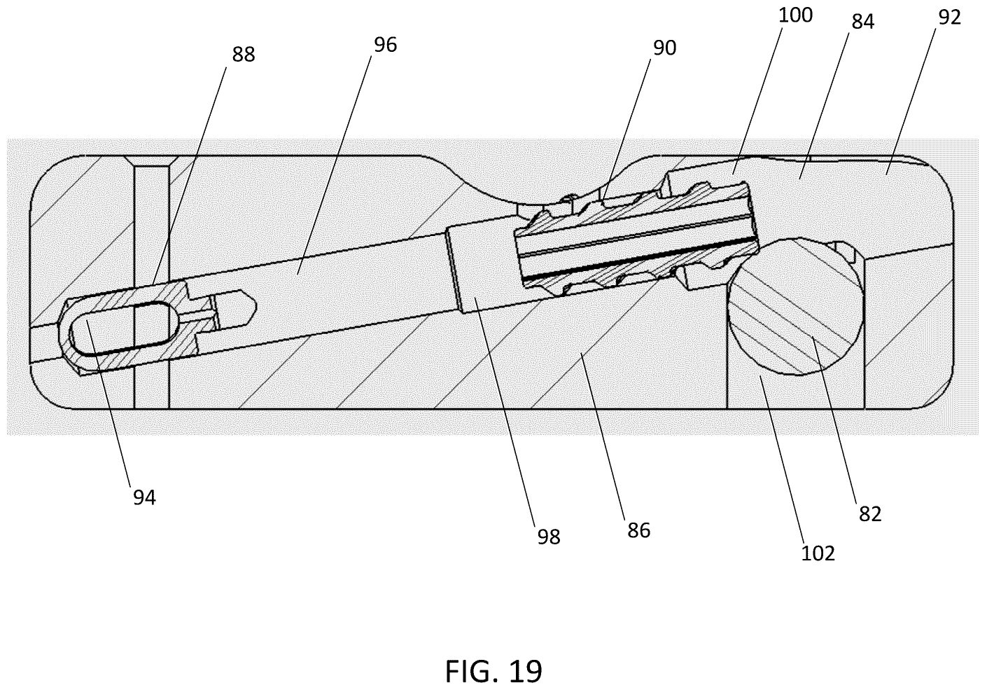

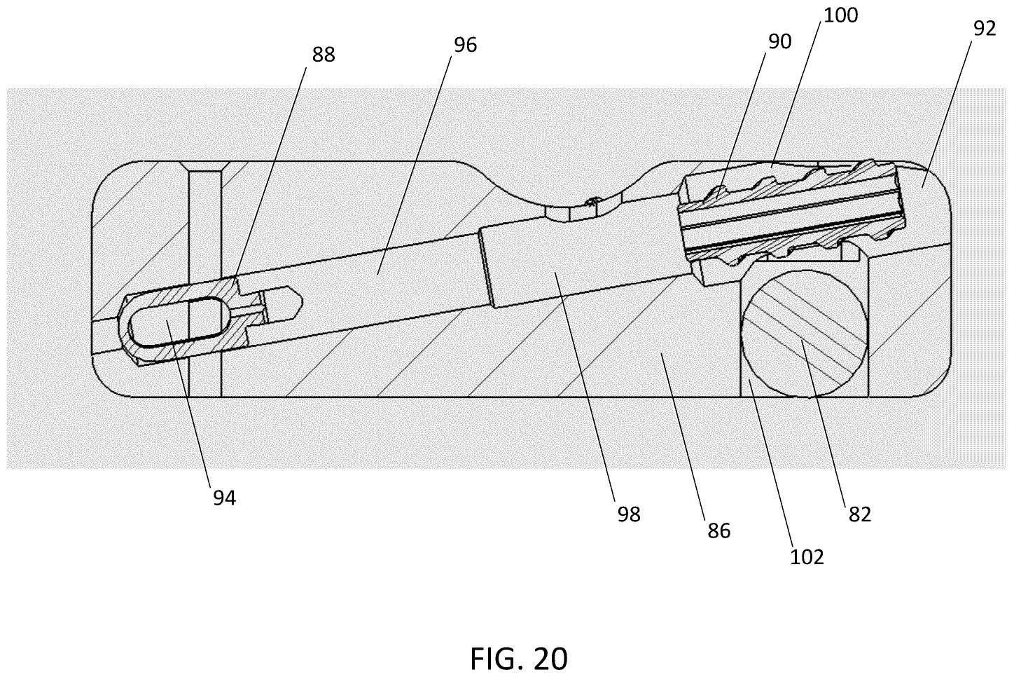

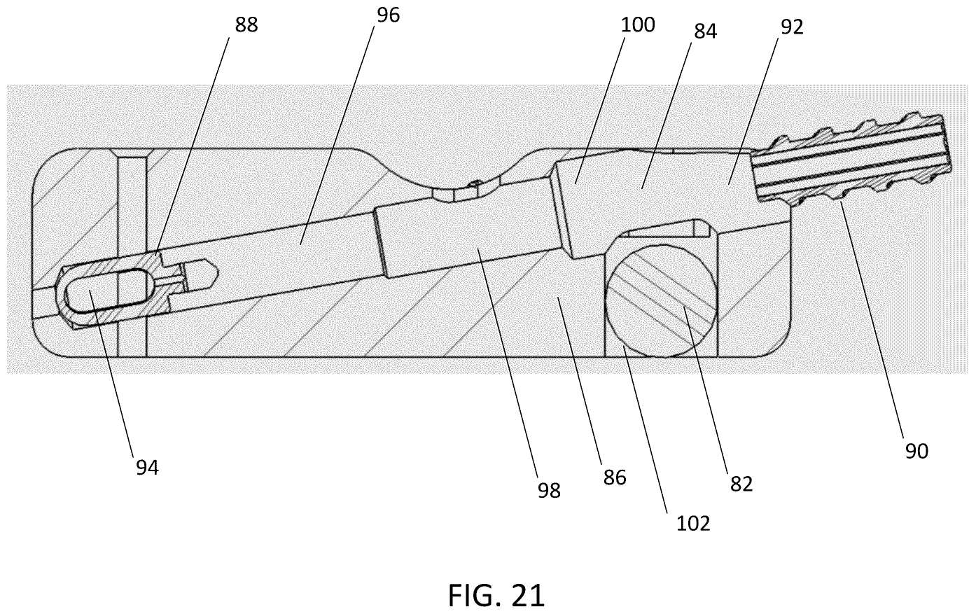

Unlike the earlier examples, the orthopedic device caddy in the example of FIGS. 17-26 includes movable stops 82 in the elongated openings 84. Each movable stop 82 is movable between a blocking position (shown in FIGS. 18 and 19) and a non-blocking position (shown in FIGS. 20 and 21). When the movable stop 82 is in the blocking position, the movable stop 82 is positioned to obstruct at least partially the elongated opening 84 such that the second orthopedic device 90 cannot move fully out to the second portion 98 of the elongated opening 84. This prevents the second orthopedic devices 90 (and thus the first orthopedic devices 88) from becoming unintentionally dislodged from the orthopedic device caddy. When the movable stop 82 is in the non-blocking position, the movable stop 82 does not prevent the second orthopedic device 90 (and thus the first orthopedic device 88) from moving fully out of the second portion of the elongated opening 98.

As shown in FIGS. 19 and 20, the movable stop 82 is located at the third portion 100 of the elongated opening 84. When the movable stop 82 is in the blocking position it extends further into the third portion 100 of the elongated opening 84 than when the movable stop 82 is I the non-blocking position.

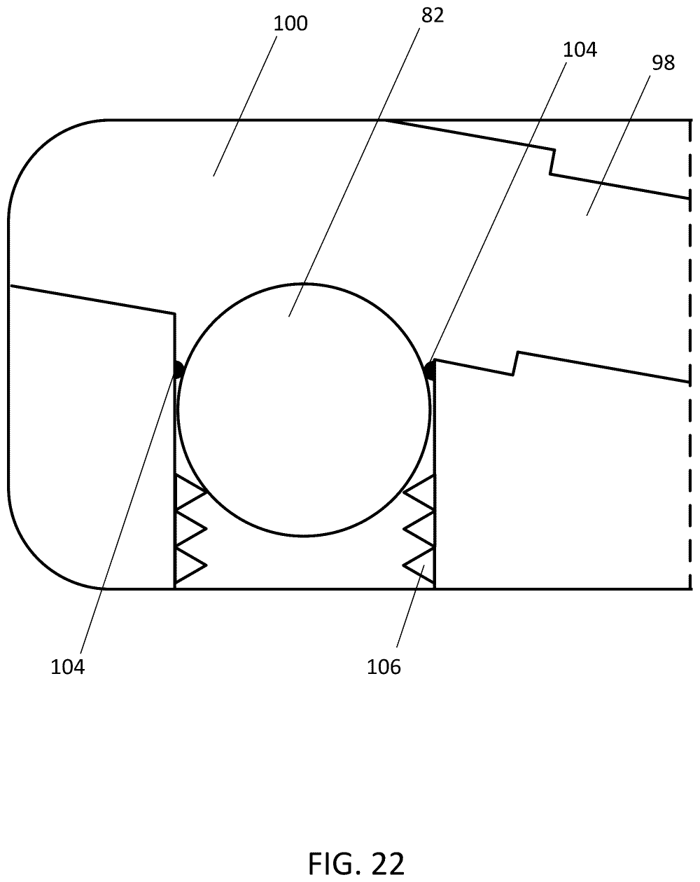

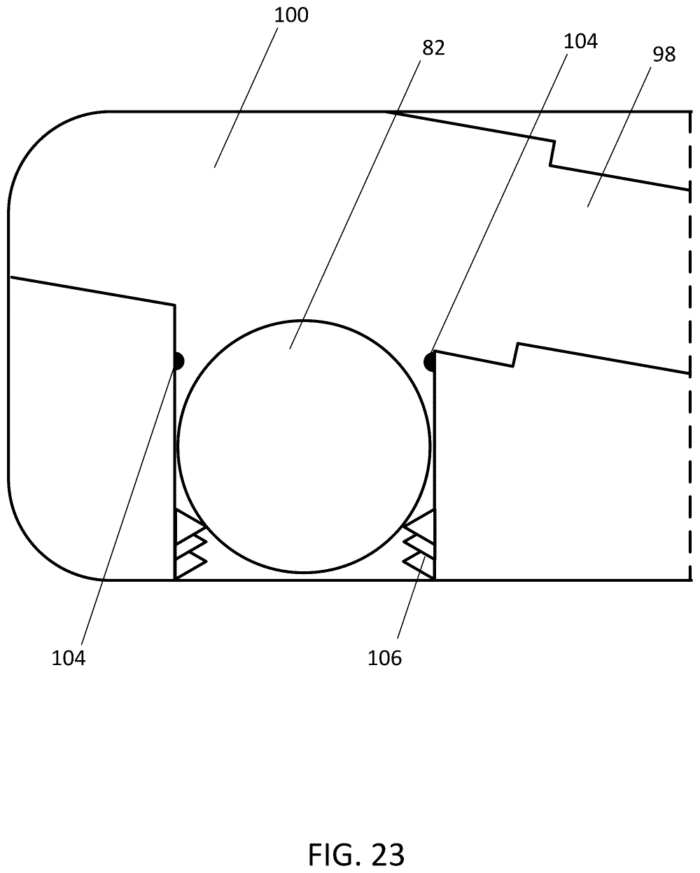

FIGS. 22 and 23 show the movable stop 82 and related parts of the orthopedic device caddy in further detail. FIG. 22 shows the movable stop 82 in the blocking position. FIG. 23 shows the movable stop 82 in the non-blocking position. The movable stop 82 (in this example a ball) is located in a cavity 102 that intersects with the third portion 100 of the elongated opening 84. A shoulder 104 in the cavity 102 prevents the movable stop 82 from moving fully out of the cavity 102. A resilient member 106 (e.g. a spring) biases the movable stop 82 to the blocking position. When the movable stop 82 is in the blocking position as shown in FIG. 22, the resilient member 106 is in an extended configuration and pushes the movable stop 82 against the shoulder 104. When the movable stop 82 is in the non-blocking position as shown in FIG. 23 the resilient member 106 is in a compressed configuration and the movable stop 82 is retracted further into the cavity 102. FIGS. 22 and 23 illustrate just one example of how a movable stop can be biased to a blocking position, other configurations are also possible.

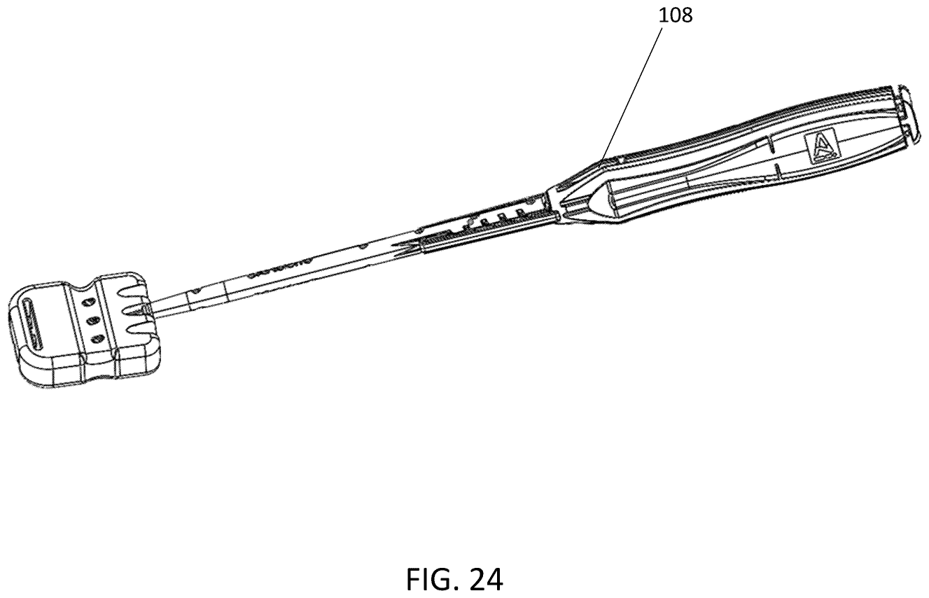

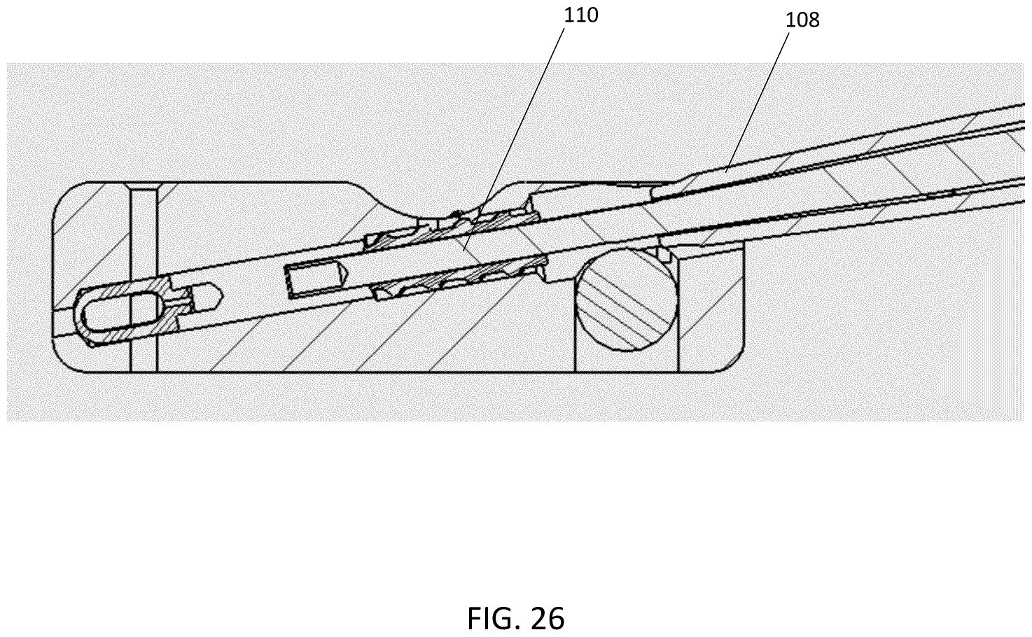

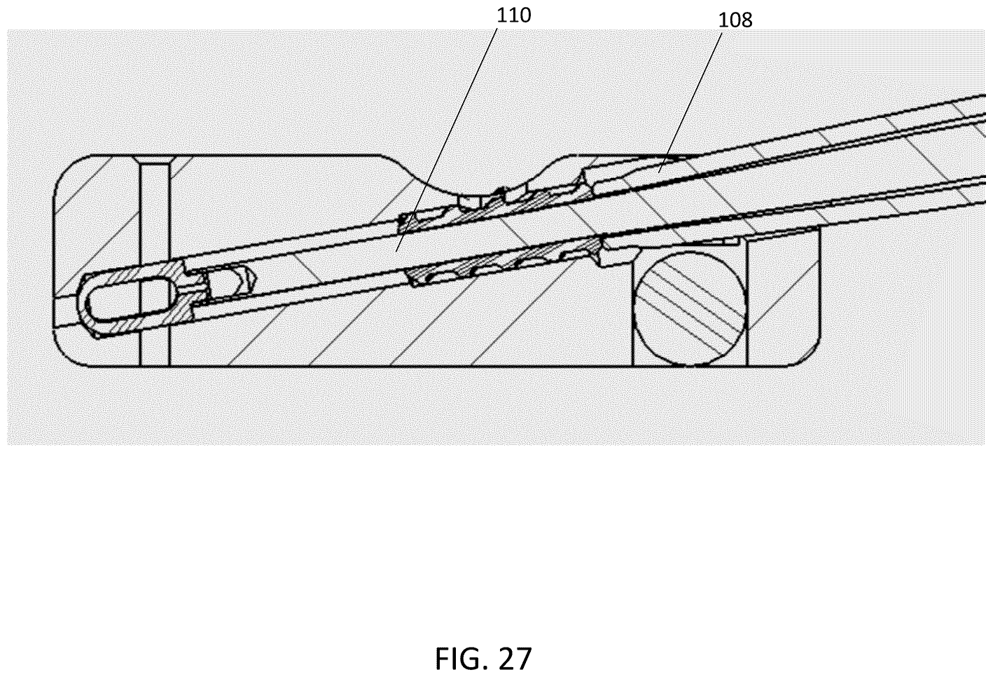

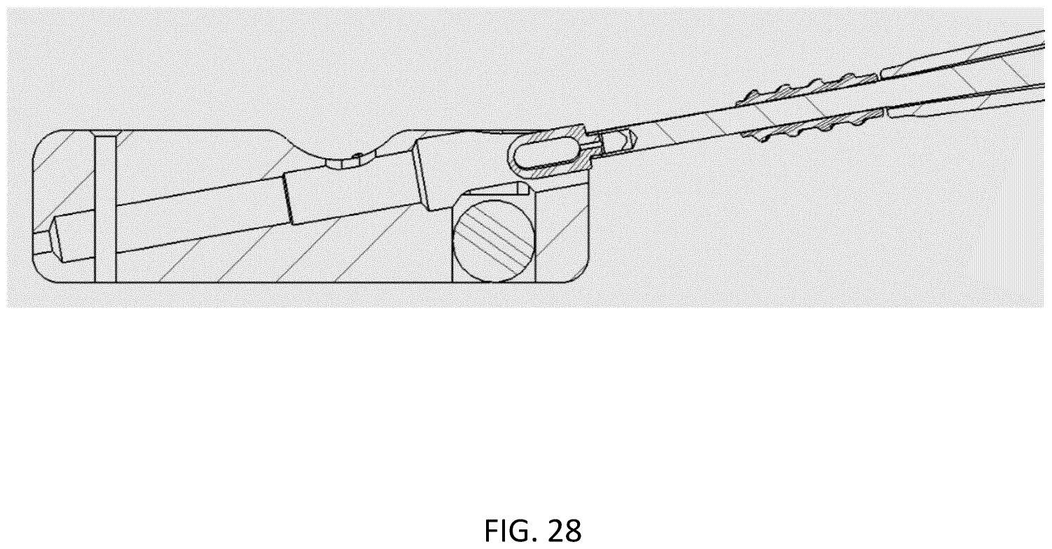

The movable stop 82 may be moved from the blocking position to the non-blocking position by insertion of an instrument into the elongated opening. FIGS. 24-28 show one example of an orthopedic instrument 108 being inserted into the elongated opening 84 of the orthopedic cavity. The orthopedic instrument 108 is sized and shaped such that insertion of it into the elongated opening 84 of the caddy body 86 moves the movable stop 82 into the non-blocking position. More specifically, as shown in FIGS. 26-27 when the orthopedic instrument 108 is inserted into the elongated opening 84, a portion of the orthopedic instrument 108 fills the third portion 100 of the elongated opening 84 and pushes the movable stop 82 down in its cavity 102, compressing the resilient member 106. At the same time, an elongated instrument shaft 110 of the orthopedic instrument 108 engages the first and second orthopedic devices 88, 90. More specifically, the elongated instrument shaft 110 enters and moves through the cannulation of the second orthopedic device 90 until a distal tip of the instrument shaft 110 contacts and engages the first orthopedic device 88. Subsequently, as shown in FIG. 28, the orthopedic instrument 108 can be withdrawn from the elongated opening 84 with the first and second orthopedic devices 88, 90 retained on the elongated instrument shaft 110 of the instrument 108. As the instrument 108 is withdrawn the movable stop 82 will return to the blocking position.

Additions, deletions, substitutions, and other modifications can be made to the orthopedic device caddy, kits, and methods described above without departing from the scope or spirit of the inventions set out in the following claims.

1. An orthopedic system comprising:
(a) an orthopedic device comprising an orthopedic device engagement feature;
(b) an orthopedic instrument comprising an elongated instrument shaft and an instrument engagement feature; and
(c) an orthopedic device caddy, comprising:
(i) a caddy body;
(ii) an elongated opening extending into the caddy body, the elongated opening comprising an alignment portion extending along a proximal segment of the elongated opening, the elongated opening further comprising an orthopedic device retention portion extending along a segment of the elongated opening distal to the proximal segment;
(c) wherein the orthopedic device retention portion is configured to hold the orthopedic device;
(d) wherein the alignment portion is configured to receive a portion of the instrument shaft and align the instrument engagement feature relative to the orthopedic device engagement feature.

2. The orthopedic system of claim 1 further comprising a sterile package containing the orthopedic device, the orthopedic instrument, and the orthopedic device caddy.

3. The orthopedic system of claim 1 wherein the alignment portion is at least three times a length of the orthopedic device.

4. The orthopedic system of claim 2 wherein the alignment portion is at least twice a length of the orthopedic device.

5. The orthopedic system of claim 4 wherein an outer diameter of the portion of the instrument shaft is less than 1 mm smaller than an inner diameter of the alignment portion of the elongated opening.

6. The orthopedic system of claim 4 wherein an outer diameter of the portion of the instrument shaft is less than 0.5 mm smaller than an inner diameter of the alignment portion of the elongated opening.

7. The orthopedic system of claim 4 wherein the elongated opening extends into the caddy body at a non-perpendicular and non-parallel angle relative to a planar face of the caddy body.

8. The orthopedic system of claim 4 wherein the caddy body is configured to allow visualization of the orthopedic device in the orthopedic device retention portion of the elongated opening.

9. The orthopedic system of claim 8 wherein the caddy body is at least partially translucent.

10. The orthopedic system of claim 8 where the caddy body comprises a visualization opening extending into the caddy body proximate the orthopedic device retention portion of the elongated opening.

11. The orthopedic system of claim 4 wherein the caddy body comprises a vent opening extending into the caddy body to the orthopedic device retention portion.

12. The orthopedic system of claim 4 wherein the caddy body comprises at least one additional opening extending into the caddy body configured to retain an additional orthopedic device.

13. The orthopedic system of claim 9 wherein the additional orthopedic device comprises an orthopedic screw.

14. The orthopedic system of claim 1 wherein:
(i) the orthopedic device comprises a first orthopedic device and a second orthopedic device;
(ii) wherein the orthopedic device retention portion of the orthopedic device caddy further comprises a first orthopedic device retention portion of the elongated opening and a second orthopedic device retention portion of the elongated opening spaced from the first orthopedic device retention portion along the elongated opening;
(iii) wherein the first orthopedic device retention portion is configured to hold the first orthopedic device; and
(iv) wherein the second orthopedic device retention portion is configured to hold the second orthopedic device.

15. The orthopedic system of claim 14 wherein the first orthopedic device retention portion is distal to the alignment portion and wherein the second orthopedic device retention portion is either proximal to or in the alignment portion.

16. The orthopedic system of claim 15 wherein the second orthopedic device is cannulated and configured to receive the elongated instrument shaft of the orthopedic instrument such that the elongated instrument shaft extends through the cannulation of the second orthopedic device.

17. The orthopedic system of claim 15 wherein the caddy body further comprises a port extending into the caddy body proximate the second orthopedic device retention portion.

18. The orthopedic system of claim 17 wherein the second orthopedic device is physically manipulable through the port when the second orthopedic device is located in the second orthopedic device retention portion.

19. An orthopedic system comprising an orthopedic device caddy, the orthopedic device caddy comprising:
(a) a caddy body;
(b) an elongated opening extending into the caddy body, the elongated opening comprising an alignment portion extending along a proximal segment of the elongated opening, the elongated opening further comprising an orthopedic device retention portion extending along a segment of the elongated opening distal to the proximal segment;
(c) wherein the orthopedic device retention portion is configured to hold an orthopedic device, the orthopedic device comprising an orthopedic device engagement feature;
(d) wherein the alignment portion is configured to align an instrument engagement feature of an orthopedic instrument relative to the orthopedic device engagement feature.

20. The orthopedic system of claim 19 wherein the alignment portion is at least three times the length of the orthopedic device retention portion.

21. The orthopedic system of claim 19 wherein the alignment portion is at least twice the length of the orthopedic device retention portion.

22. The orthopedic system of claim 21 wherein the elongated opening extends into the caddy body at a non-perpendicular and non-parallel angle relative to a planar face of the caddy body.

23. The orthopedic system of claim 21 wherein the caddy body is configured to allow visualization of the orthopedic device in the orthopedic device retention portion of the elongated opening.

24. The orthopedic system of claim 21 wherein the caddy body comprises at least one additional opening extending into the caddy body configured to retain an additional orthopedic device.

25. An orthopedic system comprising an orthopedic device caddy, the orthopedic device caddy comprising:
(a) a caddy body;
(b) an elongated opening extending into the caddy body from a proximal end to a distal end, the elongated opening comprising a first portion and a second portion proximal to the first portion that is wider than the first portion;
(c) a first orthopedic device removably positioned in the first portion of the elongated opening; and
(d) a second orthopedic device removably positioned in the second portion of the elongated opening.

26. The orthopedic system of claim 25 wherein the second orthopedic device is cannulated, with the cannulation extending from a proximal end of the second orthopedic device to a distal end of the second orthopedic device.

27. The orthopedic system of claim 26 wherein, when the first and second orthopedic devices are removably positioned in the first and second portions of the elongated opening, the distal end of the second orthopedic device is spaced apart from a proximal end of the first orthopedic device.

28. The orthopedic system of claim 25 further comprising a movable stop movable between a blocking position and a non-blocking position, wherein, when the movable stop is in the blocking position, the movable stop is positioned to at least partially obstruct the elongated opening such that the second orthopedic device cannot move fully out of the second portion; and wherein, when the movable stop is in the non-blocking position, the movable stop does not prevent the second orthopedic device from moving fully out of the second portion.

29. The orthopedic system of claim 28 wherein in the blocking position the movable stop extends further into the elongated opening than in the non-blocking position.

30. The orthopedic system of claim 28 wherein the elongated opening comprises a third portion proximal to the second portion and wider than the second portion, wherein the movable stop is located at the third portion of the elongated opening.

31. The orthopedic system of claim 28 wherein the movable stop is biased to the blocking position by a resilient member.

32. The orthopedic system of claim 31 wherein the movable stop is a movable ball.

33. The orthopedic system of claim 28 further comprising an orthopedic instrument comprising an elongated instrument shaft, wherein the orthopedic instrument is sized and shaped such that insertion of the orthopedic instrument into the elongated opening of the caddy body engages the first and second orthopedic devices to the elongated instrument shaft and moves the movable stop to the non-blocking position.
